(19) United States
(12) Plant Patent Application Publication
(10) Pub. No.: US 2004/0143877 P1
(43) Pub. Date: Jul. 22, 2004

(54) Botanical/commercial classification viola cornuta/viola plant varietal denomination cv. ' SUNVIOKI'

(76) Inventors: Kiyoshi Miyazaki, Hikone (JP); Naoto Takamura, Omihachiman (JP); Yukihiro Ikeda, Akashi (JP)

Correspondence Address: CHRISTIE, PARKER & HALE, LLP, P.O. BOX 7068, PASADENA, CA, 91109-7068, US

(21) Appl. No.: 10/611,807

(22) Filed: Jun. 30, 2003

(30) Foreign Application Priority Data
Dec. 26, 2002 (JP) 15366

(51) Int. Cl. A01H 5/00

(52) U.S. Cl. PLT/323

(57) ABSTRACT

Disclosed herein is new Viola cornuta plant named ‘SUNVIOKI’, which is the first vegetative propagated variety, which was developed by specific hybridization, between ‘9V-39a’, and ‘Alpine Sun’ has a spreading habit with very long stem. The plant height is low. The branching is abundant. Flowers are vertically oblong and very small. The color of flower is brilliant greenish yellow with vivid yellow eye. The plant has a high resistance to cold, pests and diseases.

### BOTANICAL/COMMERCIAL CLASSIFICATION

Viola cornuta/Viola Plant

### VARIETAL DENOMINATION

cv. ‘Sunvioki’

### BACKGROUND OF THE VARIETY

The genus of Viola is Violaceae plants and has about 500 species of annuals, biennials, evergreen, and semi-evergreen and deciduous perennials found in varied habitats in temperature regions worldwide.

Each flower has 5 petals a spurred lower petal, 2 lateral petals and 2 upward-facing upper petals.

Many cultivars within the genus are informally referred to as garden pansies, violas they are all derived from the complex hybridization of V. tricolour, V.lutea, V. cornuta, and other species.

Viola cornuta has played an important role in the development of garden violas and violettas as a result of cross breeding with the garden pansy which V.wittorockiana.

Viola cornuta is stem prostrate, Leaves is narrow to broad ovate, base cordate to truncate, margin deeply segmented.

The female parent used in the crossing of ‘SUNVIOKI’ was ‘9V-39a’ which is our breeding line and grown at Omi R&D Center of Suntory Flowers Ltd.

The pollen parent used in the crossing of ‘SUNVIOKI’ was ‘Alpine Sun’, whose seeds were bought from Daiichi Seed Co., Ltd. (Japan).

### BREEDING PROCESS

First of all, crossing between ‘9V-39a’ as female parent and ‘Alpine Sun’ as male parent was conducted at the Omi R&D Center of Suntory Flowers Ltd. in April 2000. In May, some seeds were obtained from the crossing. The seeds were sown and grown there. In August, 3 seedlings were selected in view of having highly spreading habit with yellow petals from 20 seedlings obtained, and propagated by cutting. Those seedlings were carried out a trial by flower potting and bedding from October 2001, at the Omi R&D Center of Suntory Flowers Ltd. Finally one variety was selected in view of having long blooming time. The botanical characteristics of that plant were then examined, using similar variety ‘Alpine Spring’ and ‘Violetto Lune’ for comparison. As a result, it was concluded that this Viola cornuta plant is distinguishable from any other variety, whose existence is known to us, and uniform and stable in its characteristics. Then the new variety of Viola cornuta plant was named ‘SUNVIOKI’.

Those of Viola cornuta plants, ‘9V-39a’ and ‘Alpine Sun’ are presently maintained at the Omi R&D Center of Suntory Flowers Ltd., located at 863-1, Aza-Iketani, Omori-cho, Yokaichi-shi, Shiga-ken, Japan.

This new and distinct variety of Viola cornuta plant, ‘SUNVIOKI’, was asexually reproduced by tissue culture at the aforementioned the Omi R&D Center of Suntory Flowers Ltd., and the homogeneity and stability thereof were confirmed.

### DESCRIPTION

In the following description, the color-coding is in accordance with the Horticultural Color Chart of The Royal Horticultural Society, London, England (R.H.S. Color Chart).

The botanical characteristics of the female parent plant ‘9V-39a’ used in the crossing of ‘SUNVIOKI’ are as follows.

Plant:

Stemmed or non-stemmed.—Stemmed.

Growth habit.—Spreading.

Height.—9 cm.

Plant cover.—49 cm.

Stem:

Length.—36 cm.

Branching.—Much.

Pubescence.—Present.

Leaf:

Overal form.—Long oblong.

Apex form.—Acute.

Base form.—Acute.

Serrated, lobed or not.—Serrated or lobed.

Margin.—Serrated or slightly lobed.

Length.—1.9 cm.

Width.—1.2 cm.

Color of upper surface.—Grayish olive green (R.H.S. 137A).

Color of under surface.—Moderate yellow green (R.H.S. 137C).

Variegation.—Absent.

Leaf luster.—Lustrous.

Vein.—Distinct.

Petiole length.—1.4 cm.

Stipule length.—1.8 cm.

Flower:

Peduncle length.—7.5 cm.

Peduncle thickness.—Thin.

Peduncle color.—Strong yellow green (R.H.S. 144B).

Flower form.—Vertically oblong.

Flowering direction.—Upward.

Longitudinal diameter.—3.2 cm.

Horizontal diameter.—3.0 cm.

Ruffle of petal margin.—Weakly ruffled.

Petal curving.—Very weak.

Petal number.—Single.

Flower color.—Self-colored.

Upper petal:

Form.—Round.

Size.—1.6×1.6 cm.

Petal color.—Self-colored.

Ground color.—Pale yellow green (R.H.S. 157A).

Blotch.—Absent.

Side petal:

Form.—Ovate.

Size.—1.3×1.5 cm.

Petal color.—Self-colored.

Ground color.—Pale yellow green (R.H.S. 157A).

Blotch.—Absent.

Lower petal:

Form.—Cordate.

Size.—1.9×1.9 cm.

Petal color.—Self-colored.

Ground color.—Pale yellow green (R.H.S. 157A).

Blotch.—Absent.

Eye color.—Vivid reddish yellow (R.H.S. 14A).

Eye size.—Medium.

Thickness of petal.—Medium.

Spur length.—Longer than the sepal appendage.

Sepal.—Short.

Number of flowers.—Many.

Seed:

Color.—Deep brown.

Physical property:

Flowering time.—Late.

Fragrance.—Faint or weak.

Resistance:

Rain.—Strong.

Cold.—Strong.

Heat.—Medium.

Disease.—Strong.

Pest.—Strong.

And the botanical characteristics of the male parent plant ‘Alpine Sun’, used in the crossing of ‘SUNVIOKI’ are as follows.

Plant:

Stemmed or non-stemmed.—Stemmed.

Growth habit.—Spreading.

Height.—8 cm.

Plant cover.—35 cm.

Stem:

Length.—25 cm.

Branching.—Much.

Pubescence.—Present.

Leaf:

Overal form.—Long oblong.

Apex form.—obtuse.

Base form.—Acute.

Serrated, lobed or not.—Serrated or lobed.

Margin.—Serrated or slightly lobed.

Length.—2.1 cm.

Width.—1.0 cm.

Color of upper surface.—Grayish olive green (R.H.S. 137A).

Color of under surface.—Moderate yellow green (R.H.S. 137C).

Variegation.—Absent.

Leaf luster.—Matte.

Vein.—Apparent.

Petiole length.—2.2 cm.

Stipule length.—2.1 cm.

Flower:

Peduncle length.—8.0 cm.

Peduncle thickness.—Thin.

Peduncle color.—Strong yellow green (R.H.S. 144B).

Flower form.—Vertically oblong.

Flowering direction.—Upward.

Longitudinal diameter.—3.3 cm.

Horizontal diameter.—2.5 cm.

Ruffle of petal margin.—Flat.

Petal curving.—Weak.

Petal number.—Single.

Flower color.—Self-colored.

Upper petal:

Form.—Round.

Size.—1.1×1.0 cm.

Petal color.—Self-colored.

Ground color.—Vivid yellow (R.H.S. 9B).

Blotch.—Absent.

Side petal:

Form.—Ovate.

Size.—1.2×1.0 cm.

Petal color.—Self-colored.

Ground color.—Vivid yellow (R.H.S. 9B).

Blotch.—Absent.

Lower petal:

Form.—Cordate.

Size.—1.4×1.3 cm.

Petal color.—Self-colored.

Ground color.—Vivid yellow (R.H.S. 9A).

Blotch.—Absent.

Eye color.—Vivid yellow (R.H.S. 13A).

Eye size.—Small.

Thickness of petal.—Thin.

Spur length.—Longer than the sepal appendage.

Sepal.—Short.

Number of flowers.—Many.

Seed:

Color.—Deep brown.

Physical property;

Flowering time.—Late.

Fragrance.—Absent.

Resistance:

Rain.—Medium.

Cold.—Strong.

Heat.—Medium.

Disease.—Strong.

Pest.—Strong.

The botanical characteristics of the similar variety ‘Alpine Spring’ used for examination as a comparison are as follows.

Plant:

Stemmed or non-stemmed.—Stemmed.

Growth habit.—Spreading.

Height.—8 cm.

Plant cover.—35 cm.

Stem:

Length.—25 cm.

Branching.—Much.

Pubescence.—Present.

Leaf:

Overal form.—Long oblong.

Apex form.—Obtuse.

Base form.—Acute.

Serrated, lobed or not.—Serrated or lobed.

Margin.—Serrated or slightly lobed.

Length.—2.1 cm.

Width.—1.0 cm.

Color of upper surface.—Grayish olive green (R.H.S. 137A).

Color of under surface.—Moderate yellow green (R.H.S. 137C).

Variegation.—Absent.

Leaf luster.—Matte.

Vein.—Apparent.

Petiole length.—2.2 cm.

Stipule length.—2.1 cm.

Flower:

Peduncle length.—8.0 cm.

Peduncle thickness.—Thin.

Peduncle color.—Strong yellow green (R.H.S. 144B).

Flower form.—Vertically oblong.

Flowering direction.—Upward.

Longitudinal diameter.—3.3 cm.

Horizontal diameter.—2.5 cm.

Ruffle of petal margin.—Flat.

Petal curving.—Weak.

Petal number.—Single.

Flower color.—Self-colored.

Upper petal:

Form.—Round.

Size.—1.1×1.0 cm.

Petal color.—Self-colored.

Ground color.—Brilliant yellow green (R.H.S. 2B).

Blotch.—Absent.

Side petal:

Form.—Ovate.

Size.—1.2×1.0 cm.

Petal color.—Self-colored.

Ground color.—Vivid yellow (R.H.S. 9B).

Blotch.—Absent.

Vein color.—Dark reddish purple (R.H.S. 79A).

Lower petal:

Form.—Cordate.

Size.—1.4×1.3 cm.

Petal color.—Self-colored.

Ground color.—Vivid yellow (R.H.S. 9A).

Blotch.—Absent.

Vein color.—Dark reddish purple (R.H.S. 79A).

Eye color.—Vivid yellow (R.H.S. 13A).

Eye size.—Small.

Thickness of petal.—Thin.

Spur length.—Longer than the sepal appendage.

Sepal.—Short.

Number of flowers.—Many.

Seed:

Color.—Deep brown.

Physical property:

Flowering time.—Late.

Fragrance.—Absent.

Resistance:

Rain.—Medium.

Cold.—Strong.

Heat.—Medium.

Disease.—Strong.

Pest.—Strong.

The botanical characteristics of the similar variety ‘Violetto Lune’ used for examination as a comparison are as follows.

Plant:

Stemmed or non-stemmed.—Stemmed.

Growth habit.—Spreading.

Height.—6 cm.

Plant cover.—40 cm.

Stem:

Length.—26 cm.

Branching.—Much.

Pubescence.—Present.

Leaf:

Overal form.—Long oblong.

Apex form.—Obtuse.

Base form.—Obtuse.

Serrated, lobed or not.—Serrated or lobed.

Margin.—Serrated or slightly lobed.

Length.—2.2 cm.

Width.—1.6 cm.

Color of upper surface.—Grayish olive green (R.H.S. 137B).

Color of under surface.—Moderate yellow green (R.H.S. 137C).

Variegation.—Absent.

Leaf luster.—Matte.

Vein.—Apparent.

Petiole length.—1.3 cm.

Stipule length.—2.0 cm.

Flower:

Peduncle length.—9.0 cm.

Peduncle thickness.—Thin.

Peduncle color.—Strong yellow green (R.H.S. 144B).

Flower form.—Vertically oblong.

Flowering direction.—Horizontal.

Longitudinal diameter.—3.4 cm.

Horizontal diameter.—2.9 cm.

Ruffle of petal margin.—Flat.

Petal curving.—Weak.

Petal number.—Single.

Flower color.—Self-colored.

Upper petal:

Form.—Round.

Size.—1.9×1.9 cm.

Petal color.—Self-colored.

Ground color.—Vivid yellow (R.H.S. 9A).

Blotch.—Absent.

Side petal:

Form.—Round.

Size.—1.2×1.1 cm.

Petal color.—Self-colored.

Ground color.—Vivid yellow (R.H.S. 9A).

Blotch.—Absent.

Lower petal:

Form.—Cordate.

Size.—1.8×1.1 cm.

Petal color.—Self-colored.

Ground color.—Vivid yellow (R.H.S. 12A).

Blotch.—Absent.

Eye color.—Vivid yellow (R.H.S. 13A).

Eye size.—Small.

Thickness of petal.—Thin.

Spur length.—Longer than the sepal appendage.

Sepal.—Short.

Number of flowers.—Many.

Seed:

Color.—Deep brown.

Physical property:

Flowering time.—Late.

Fragrance.—Absent.

Resistance:

Rain.—Medium.

Cold.—Strong.

Heat.—Medium.

Disease.—Strong.

Pest.—Strong.

### SUMMARY OF THE NEW VARIETY

The new variety of viola plant named ‘SUNVIOKI’, which is the first vegetative propagated variety, has a spreading plant habit with very long epigeal stem. The plant height is low and the branching is abundant. The flowers of the new variety are vertically oblonged and very small, and the color of flower is brilliant greenish yellow with vivid yellow eye. The new variety has a high resistance to cold, pests and diseases.

### BRIEF DESCRIPTION OF THE DRAWING

FIG. 1 is a photograph giving a partial view of the new variety of Viola cornuta plant ‘SUNVIOKI’ planted in pots.

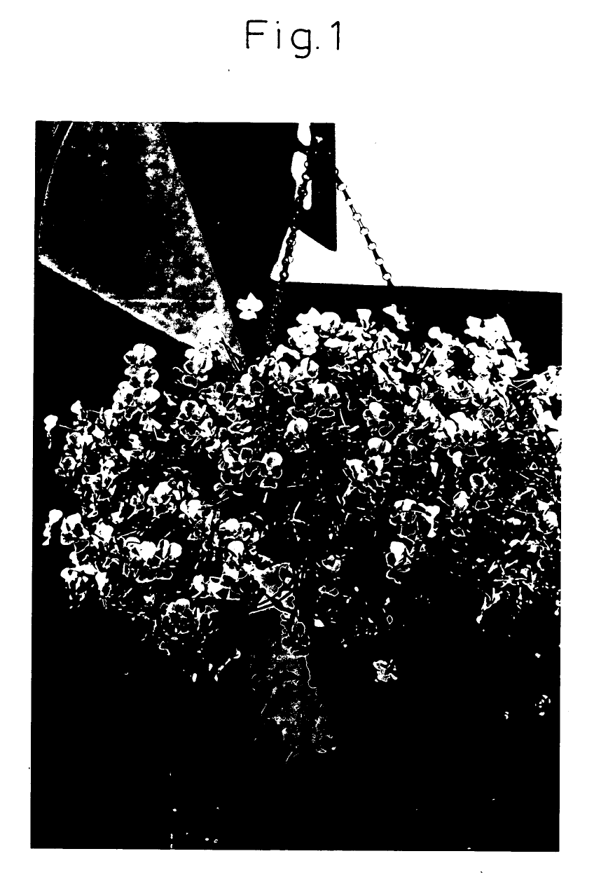

FIG. 2 is a photograph of flowers of the new variety of Viola cornuta plant ‘SUNVIOKI’.

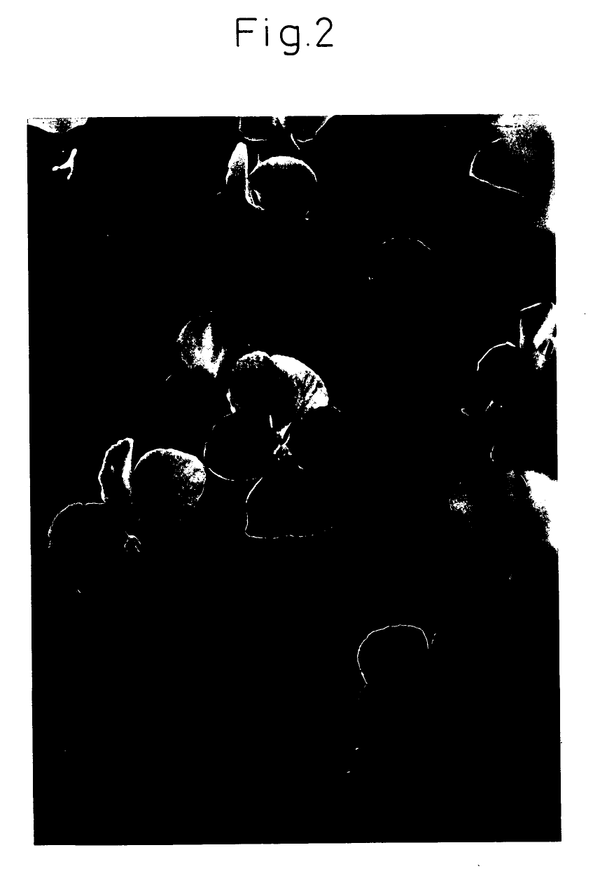

### DESCRIPTION OF THE VARIETY

The botanical characteristics of this new and distinct variety of Viola cornuta plant named ‘SUNVIOKI’ are as follows.

Plant:

Stemmed or non-stemmed.—Stemmed.

Growth habit.—Spreading.

Height.—8 cm.

Plant cover.—75 cm.

Stem:

Length.—45 cm.

Branching.—abundant.

Pubescence.—Present.

Leaf:

Overal form.—Long oblong.

Apex form.—Obtuse.

Base form.—Acute.

Serrated, lobed or not.—Serrated or lobed.

Margin.—Serrated or slightly lobed.

Length.—3.5 cm.

Width.—1.7 cm.

Color of upper surface.—Grayish olive green (R.H.S. 137A).

Color of under surface.—Moderate yellow green (R.H.S. 137C).

Variegation.—Absent.

Leaf luster.—Matte.

Vein.—Apparent.

Petiole length.—2.5 cm.

Stipule length.—1.5 cm.

Flower:

Peduncle length.—10.0 cm.

Peduncle thickness.—Thin.

Peduncle color.—Strong yellow green (R.H.S. 144A).

Flower form.—Vertically oblong.

Flowering direction.—Upward.

Longitudinal diameter.—3.5 cm.

Horizontal diameter.—2.6 cm.

Ruffle of petal margin.—Weakly ruffled.

Petal curving.—Weak.

Petal number.—Single.

Flower color.—Self-colored.

Upper petal:

Form.—Ovate.

Size.—1.8×1.3 cm.

Petal color.—Self-colored.

Ground color.—Brilliant greenish yellow (R.H.S. 5B).

Blotch.—Absent.

Side petal:

Form.—Ovate.

Size.—1.3×1.1 cm.

Petal color.—Self-colored.

Ground color.—Brilliant greenish yellow (R.H.S. 5A).

Blotch.—Absent.

Lower petal:

Form.—Cordate.

Size.—1.7×1.8 cm.

Petal color.—Self-colored.

Ground color.—Brilliant greenish yellow (R.H.S. 5A).

Blotch.—Absent.

Eye color.—Vivid yellow (R.H.S. 13A).

Eye size.—Small.

Thickness of petal.—Thin.

Spur length.—Longer than the sepal appendage.

Sepal.—Short.

Number of flowers.—Many.

Seed:

Color.—Deep brown.

Physical property:

Flowering time.—Early.

Fragrance.—Faint or weak.

Resistance:

Rain.—Medium.

Cold.—Strong.

Heat.—Medium.

Disease.—Strong.

Pest.—Strong.

This new variety of Viola cornuta plant ‘SUNVIOKI’ is most suitable for flower bedding and potting, hanging basket and ground cover.

### COMPARISON WITH SIMILAR VARIETY

The new variety of Viola cornuta plant ‘SUNVIOKI’ is different from a similar variety, ‘Alpine Spring’ in following points,

1. The plant width of ‘SUNVIOKI’ is wider than that of ‘Alpine Spring’.

2. ‘SUNVIOKI’ has abundant branching compared to ‘Alpine Spring’ which has less branching.

3. The ruffle of petal margin of ‘SUNVIOKI’ is weakly ruffled while that of ‘Alpine Spring’ is flat.

4. ‘SUNVIOKI’ has self-colored flower while ‘Alpine Spring’ has multi-colored one.

5. The color of upper petal of ‘SUNVIOKI’ is R.H.S.5B while that of ‘Alpine Spring’ is R.H.S.2B.

6. The color of side petal of ‘SUNVIOKI’ is R.H.S.5A while that of ‘Alpine Spring’ is R.H.S.9B with R.H.S.79A vein.

7. The color of lower petal of ‘SUNVIOKI’ is R.H.S.5A while that of ‘Alpine Spring’ is R.H.S.9A with R.H.S.79A vein.

8. The flowering time of ‘SUNVIOKI’ is early while that of ‘Alpine Spring’ is late.

The new variety of Viola cornuta plant ‘SUNVIOHO’ is different from a similar variety, ‘Violetto Lune’ in following points,

1. ‘SUNVIOKI’ has abundant branching compared to ‘Violetto Lune’ which has less branching.

2. The leaf color of upper surface of ‘SUNVIOKI’ is R.H.S.137A while that of ‘Vioetto Lune’ is R.H.S.137B.

3. The color of upper petal of ‘SUNVIOKI’ is R.H.S.5B while that of ‘Violetto Lune’ is R.H.S.9A.

4. The color of side petal of ‘SUNVIOKI’ is R.H.S.5A while that of ‘Violetto Lune’ is R.H.S.9A.

5. The color of lower petal of ‘SUNVIOKI’ is R.H.S.5A while that of ‘Violetto Lune’ is R.H.S.12A.

6. The flowering time of ‘SUNVIOKI’ is early while that of ‘Violetto Lune’ is late.

1. A new and distinct variety of Viola cornuta plant named ‘SUNVIOKI’, substantially as herein illustrated and described, characterized particularly as to novelty by (A) being a spreading plant habit having very long epigeal stem, (B) the plant height is low (C) the branching is abundant, (D) flowers which are vertically oblonged and very small, the color of flower is brilliant greenish yellow with vivid yellow eye and (E) a high resistance to cold, pests and diseases.
